(19) United States
(12) Patent Application Publication
(10) Pub. No.: US 2024/0100300 A1
(43) Pub. Date: Mar. 28, 2024

(54) FLUID SHIELD ASSEMBLIES AND FLOW RESTRICTION DEVICES WITH FLUID SHIELD ASSEMBLIES

(71) Applicant: Carefusion 303, Inc., San Diego, CA (US)

(72) Inventors: Alireza Salehi Borojerdi, Aliso Viejo, CA (US); Mohammed Mehtab Khan, Whitefield Bangalore (IN); Niranjan Lekkala, Bangalore (IN); Sudharshan Mundadishetty, Udupi Dist. (IN); Prabhath Kakaraparthi, Nellore (IN)

(21) Appl. No.: 17/953,892

(22) Filed: Sep. 27, 2022

(51) Int. Cl. A61M 25/00; A61M 25/01; A61M 39/02; A61M 39/10

(52) U.S. Cl. CPC A61M 25/0097; A61M 25/01; A61M 39/02; A61M 39/10; A61M 2039/1033

(57) ABSTRACT

A flow restriction device includes a proximal housing configured to couple to a fluid collection device, a distal housing configured to couple to a catheter assembly, an intermediate housing interposed between the proximal housing and the distal housing and an internal fluid channel extending transversely therethrough. The flow restriction device also includes a fluid shield assembly having an enclosure coupled to an end of the distal housing and a split septum coupled to an end of the enclosure, the split septum comprising a flexible material with a centrally disposed slit. The fluid shield assembly provides for connection of a female connector to a male connector of the distal housing through the slit of the split septum, and to contain fluid within the enclosure and split septum upon disconnection and withdrawal of the female connector from the male connector. Blood collection systems and fluid shield assemblies are also provided.

### TECHNICAL FIELD

The present disclosure generally relates to intravenous (IV) blood draw from a patient, and particularly to systems and methods to reduce hemolysis in an over-the-needle peripheral IV catheter (PIVC) blood draw whilst reducing blood spillage.

### BACKGROUND

Catheters are commonly used for a variety of infusion therapies. For example, catheters may be used for infusing fluids, such as normal saline solution, various medicaments, and total parenteral nutrition, into a patient. Catheters may also be used for withdrawing blood from the patient.

A common type of catheter is a PIVC. As its name implies, the over-the-needle catheter may be mounted over an introducer needle having a sharp distal tip. A catheter assembly may include a catheter hub, the catheter extending distally from the catheter hub, and the introducer needle extending through the catheter. The catheter and the introducer needle may be assembled so that the distal tip of the introducer needle extends beyond the distal tip of the catheter with the bevel of the needle facing up away from skin of the patient. The catheter and introducer needle are generally inserted at a shallow angle through the skin into vasculature of the patient.

In order to verify proper placement of the introducer needle and/or the catheter in the blood vessel, a clinician generally confirms that there is “flashback” of blood in a flashback chamber of the catheter assembly. Once placement of the needle has been confirmed, the clinician may temporarily occlude flow in the vasculature and remove the needle, leaving the catheter in place for future blood withdrawal or fluid infusion.

For blood withdrawal or collecting a blood sample from a patient, a blood collection container may be used. The blood collection container may include a syringe. Alternatively, the blood collection container may include a test tube with a rubber stopper at one end. In some instances, the test tube has had all or a portion of air removed from the test tube so pressure within the test tube is lower than ambient pressure. Such a blood collection container is often referred to as an internal vacuum or a vacuum tube. A commonly used blood collection container is a VACUTAINER® blood collection tube, available from Becton Dickinson & Company.

The blood collection container may be coupled to the catheter. When the blood collection container is coupled to the catheter, a pressure in the vein is higher than a pressure in the blood collection container, which pushes blood into the blood collection container, thus filling the blood collection container with blood. A vacuum within the blood collection container decreases as the blood collection container fills, until the pressure in the blood collection container equalizes with the pressure in the vein, and the flow of blood stops.

Unfortunately, as blood is drawn into the blood collection container, red blood cells are in a high shear stress state and susceptible to hemolysis due to a high initial pressure differential between the vein and the blood collection container. Hemolysis may result in rejection and discard of a blood sample. The high initial pressure differential can also result in catheter tip collapse, vein collapse, or other complications that prevent or restrict blood from filling the blood collection container. Furthermore, blood spillage during and/or after blood draw commonly occurs.

The description provided in the background section should not be assumed to be prior art merely because it is mentioned in or associated with the background section. The background section may include information that describes one or more aspects of the subject technology.

### SUMMARY

In one or more embodiments, a flow restriction device, comprises: a proximal housing configured to couple to a fluid collection device; a distal housing configured to couple to a catheter assembly; an intermediate housing interposed between the proximal and distal housings; an internal fluid channel extending transversely through the proximal, intermediate and distal housings; and a fluid shield assembly. The fluid shield assembly comprises an enclosure coupled to an end of the distal housing and a split septum coupled to an end of the enclosure, the split septum comprising a flexible material with a centrally disposed slit. The fluid shield assembly is configured to allow connection and disconnection of a female connector to a male connector of the distal housing through the slit of the split septum, and to contain fluid within the enclosure and split septum upon disconnection and withdrawal of the female connector from the male connector.

In one or more embodiments, a blood collection system comprises: a blood collection device; a catheter assembly; and a flow restriction device fluidly coupled to the blood collection device. The flow restriction device comprises: a proximal housing coupled to a male connector of the blood collection device; a distal housing comprising a male luer connector configured to couple to a female connector of the catheter assembly; an intermediate housing interposed between the proximal housing and the distal housing; an internal fluid channel extending transversely through the proximal, intermediate and distal housings; and a fluid shield assembly. The fluid shield assembly comprises: a luer enclosure coupled to an end of the distal housing; and a split septum coupled to an end of the enclosure, the split septum comprising a flexible material with a centrally disposed slit, the split septum and the luer enclosure enclosing the male luer connector and an end portion of the distal housing. The fluid shield assembly provides for connection and disconnection of the female connector of the catheter assembly to the male luer connector of the distal housing through the slit of the split septum, and to contain excess blood within the enclosure and split septum upon disconnection and withdrawal of the female connector from the male luer connector.

In one or more embodiments, a fluid shield assembly for a male luer connector comprises: an enclosure configured to couple to an end of the male luer connector; and a split septum coupled to an end of the enclosure, the split septum comprising a flexible material with a centrally disposed slit, wherein the fluid shield assembly is configured to provide connection and disconnection of a female connector to the male luer connector through the slit of the split septum, and to contain fluid within the enclosure and split septum upon disconnection and withdrawal of the female connector from the male luer connector.

It is understood that other configurations of the subject technology will become readily apparent to those skilled in the art from the following detailed description, wherein various configurations of the subject technology are shown and described by way of illustration. As will be realized, the subject technology is capable of other and different configurations and its several details are capable of modification in various other respects, all without departing from the scope of the subject technology. Accordingly, the drawings and detailed description are to be regarded as illustrative in nature and not as restrictive.

### BRIEF DESCRIPTION OF THE DRAWINGS

The following figures are included to illustrate certain aspects of the embodiments and should not be viewed as exclusive embodiments. The subject matter disclosed is capable of considerable modifications, alterations, combinations, and equivalents in form and function, as will occur to those skilled in the art and having the benefit of this disclosure.

FIG. 1 illustrates a vascular access device including a PIVC assembly that includes a flow restriction device, in accordance with some embodiments of the present disclosure.

FIG. 2 illustrates a perspective view of the flow restriction device, in accordance with some embodiments of the present disclosure.

FIG. 3 illustrates a vascular access device including a PIVC assembly that includes a flow restriction extension set, in accordance with some embodiments of the present disclosure

FIG. 4 illustrates a perspective view of the flow restriction extension set, in accordance with some embodiments of the present disclosure.

FIG. 5 illustrates a cross-sectional perspective view of a portion of a fluid shield assembly and male luer connector, in accordance with some embodiments of the present disclosure.

FIG. 6 illustrates a top view of a fluid shield assembly, in accordance with some embodiments of the present disclosure.

FIG. 7 illustrates a bottom view of the fluid shield assembly of FIG. 6, in accordance with some embodiments of the present disclosure.

FIG. 8 illustrates a bottom perspective view of the fluid shield assembly of FIG. 6, in accordance with some embodiments of the present disclosure.

FIG. 9 illustrates a cross-sectional perspective view of the fluid shield assembly of FIG. 6, in accordance with some embodiments of the present disclosure.

FIG. 10 illustrates a perspective view of a connector assembly having a female connector coupled to a male connector and a fluid shield assembly, in accordance with some embodiments of the present disclosure.

FIG. 11 illustrates a cross-sectional perspective view of the connector assembly of FIG. 10, in accordance with some embodiments of the present disclosure.

### DETAILED DESCRIPTION

The detailed description set forth below describes various configurations of the subject technology and is not intended to represent the only configurations in which the subject technology may be practiced. The detailed description includes specific details for the purpose of providing a thorough understanding of the subject technology. Accordingly, dimensions may be provided in regard to certain aspects as non-limiting examples. However, it will be apparent to those skilled in the art that the subject technology may be practiced without these specific details. In some instances, well-known structures and components are shown in block diagram form in order to avoid obscuring the concepts of the subject technology.

It is to be understood that the present disclosure includes examples of the subject technology and does not limit the scope of the appended claims. Various aspects of the subject technology will now be disclosed according to particular but non-limiting examples. Various embodiments described in the present disclosure may be carried out in different ways and variations, and in accordance with a desired application or implementation.

Blood draw via a vascular access device has drawn increasing attention attributed to minimized needle sticks and improved operation efficiency as compared with traditional blood draw methods with venipuncture. Current blood draw using a PIVC has seen some challenges, one of the most critical is hemolysis related blood quality. In particular, with currently existing PIVC products in the market, along with the standard connection (such as a short extension set and a needleless connector), and blood collection devices (such as a Vacutainer), the shear stress exerted onto blood cells tends to be on the verge of hemolyzing.

Various embodiments of the present disclosure are directed to providing assemblies and methods to address blood spillage in PIVC blood draw with a hemolysis reduction accessory (also referred to herein as a flow restriction device) which is pre-attached to the PIVC and serves as a flow restrictor to reduce risk of hemolysis. The hemolysis-reduction accessory is advantageously compatible with PIVC placement and does not necessitate change to any of the existing operations. The hemolysis-reduction accessory of the various embodiments described herein is potentially applicable to a wide variety of PIVC products, and compatible with existing blood collection devices and infusion disposables.

Here, the flow restriction device regulates the overall flow rate of the entire fluid path as blood cells travel through. The flow restriction device can be either assembled with the PIVC or co-packaged with the PIVC. As such, there is no additional operation during catheter placement since the device has a vented lumen that enables blood flashback. The clinician may connect a blood collection device to the port of the accessory and can then draw blood to the intended volume. After blood draw, the clinician may disconnect and discard the flow restriction device and the blood collection device together. As such, this flow restriction device can be either for single blood draw or stay inline throughout indwell.

In some embodiments, the flow restriction device may be an integral connector that includes a proximal housing, a distal housing and an intermediate housing, each having an aligned fluid channel disposed axially within. Accordingly, fluid communication may be established between the proximal, distal, and intermediate housings to allow a fluid to flow from the distal housing to the proximal housing via the intermediate housing. The aligned internal fluid channels together may define a linear internal flow channel through the flow restriction device. As such, during blood collection or withdrawal from the patients, blood may flow from the patient's vein into a blood collection device via the internal flow channel of the flow restriction device.

In some embodiments, the flow restriction device may be an extension set that includes an IV tube with a male luer connection on one end and a female luer connection on the other end. Accordingly, fluid communication may be established between the male and female luer connections and the IV tube to allow a fluid to flow from the female luer connection to the male luer connection via the IV tube. As such, during blood collection or withdrawal from the patients, blood may flow from the patient's vein into a blood collection device via an internal flow channel of the flow restriction device.

During blood draw with currently existing blood draw devices, for example, blood cells may experience shear stress as they flow from the distal housing to the proximal housing. The maximum shear stress may be along the wall of the blood cell, often referred to as wall shear stress. Wall shear stress on blood cells is considered a major source of mechanical damage to blood cells causing hemolysis of the blood cells. In some embodiments, the linear internal flow channel of the flow restriction device having minimal diameter may facilitate increased flow resistance within the vascular access system to distribute the pressure differential and reduce shear stress experienced by the red blood cells of the blood. For example, the minimized diameter of the internal flow channel may provide increased resistance to flow of the blood and thereby decrease blood flow rate within the flow resistance device. Since the decreased blood flow rate causes a reduction in shear stress experienced by the red blood cells of the blood, a risk of hemolysis during blood collection may advantageously be reduced.

In some embodiments, the integral connector flow restriction device distal housing may include a male connector, such as a male luer connector, and the proximal housing may include a female connector, such as a needleless connector. In some embodiments, the extension set flow restriction device may include a male connector, such as a male luer connector, and a female connector, such as a needleless connector. In some embodiments, the flow restriction device may include a fluid shield assembly coupled to the distal housing or the male connector. For example, the fluid shield assembly may include a split septum and an enclosure to fit on the male luer connector.

Various embodiments of the present disclosure are directed to providing assemblies and methods to address blood spillage in PIVC blood draw with an extension set with a male luer connector on one end and a female luer connector on the opposing end. A standard ISO male luer connector is used to be connected to a catheter side needle-free connector, which has the potential of blood exposure from the male luer connector while disconnecting after blood draw is complete.

A blood draw device is often used with a luer lock access device (LLAD) or a syringe (e.g., the blood draw device is connected with an LLAD or a syringe and then used for a blood draw). It is typically prescribed to dispose the LLAD or the syringe and the blood draw device together to avoid blood spillage. After the blood draw, i.e., after removal of a blood filled container (e.g., Vacutainer), the next step is to disconnect the blood draw device that is filled with remaining blood. Thus, an issue is the potential blood exposure to a clinician as the clinician disconnects the blood draw device from the PIVC needle-free connector.

In some embodiments, a split septum with a small enclosure around the male luer is provided at the front of the blood draw device connector. The needle-free connector is pushed into the split septum while it is connected to the blood draw device. The split septum self-seals and prevents or minimizes left over blood from spilling from the blood draw device when the needle-free connector is removed. The septum blocks within the enclosure and avoids any spillage. The split septum may be made of silicone material and may be coupled to the luer enclosure in any suitable manner (e.g., glued, welded, overmolded). The split septum may also be coupled to the blood draw device connector at the front of the male luer. For example, the split septum may be glued to the enclosure and the enclosure may be friction fit over the front end of the male luer connector of the blood draw device, where the split septum may also be glued to the front rim of the male luer connector.

The flow restriction devices and associated blood collection systems of the various embodiments described herein additionally provide further advantages over currently existing blood collection systems. For example, add-on flow restriction devices described herein allow for hemolysis-reduction function to be integrated for PIVC blood draw. Further, the flow restriction devices described herein are compatible with PIVC placement and allow for seamless blood draw at insertion. Additionally, since the flow restriction devices are an add-on which can be easily incorporated without any changes to existing PIVC, there is minimal impact to clinical setting and operations. As another example, extension sets with a male luer connector described herein allow for safe disconnection from an existing PIVC.

FIG. 1 illustrates a vascular access device 100 including a PIVC assembly 50 that includes a flow restriction device 110, in accordance with some embodiments of the present disclosure. The flow restriction device 110 may be configured to reduce a likelihood of hemolysis during blood collection using the vascular access device 100. In some embodiments, the vascular access device 100 may include the catheter assembly 50. The catheter assembly 50 may include a catheter hub 52, which may include a distal end 54, a proximal end 56, and a lumen extending through the distal end 54 and the proximal end 56. The catheter assembly 50 may further include a catheter 58, which may be secured within the catheter hub 52 and may extend distally from the distal end 54 of the catheter hub 52. In some embodiments, the catheter assembly 50 may be a PIVC or any other suitable catheter device.

In some embodiments, the catheter assembly 50 may include or correspond to any suitable catheter assembly 50. In some embodiments, the catheter assembly 50 may be integrated and include an extension tube 60, which may extend from and be integrated with a side port 59 of the catheter hub 52. A non-limiting example of an integrated catheter assembly is the BD NEXIVA™ Closed IV Catheter system, available from Becton Dickinson and Company. In some embodiments, a proximal end of the extension tube 60 may be coupled to an adapter, such as, for example, a Y-adapter 70. In some embodiments, the flow restriction device 110 may be fluidly coupled to the Y-adapter 70, such as to a needle free connector 72.

In some embodiments, the catheter assembly 50 may be non-integrated and may not include the extension tube 60. In these and other embodiments, the flow restriction device 110 may be configured to couple to the proximal end 56 of the catheter hub 52 or another suitable portion of the catheter assembly 50. In some embodiments, the flow restriction device 110 may be coupled directly to the catheter assembly 50, eliminating the extension tube 60 and providing a compact catheter system.

In accordance with some embodiments, the flow restriction device 110 may be configured to couple to a blood collection device 40. For example, the flow restriction device 110 may include a female luer connector 113 at proximal housing 112, which may be coupled with a male luer connector 42 of the blood collection device 40. In some embodiments, the blood collection device 40 may be a LLAD.

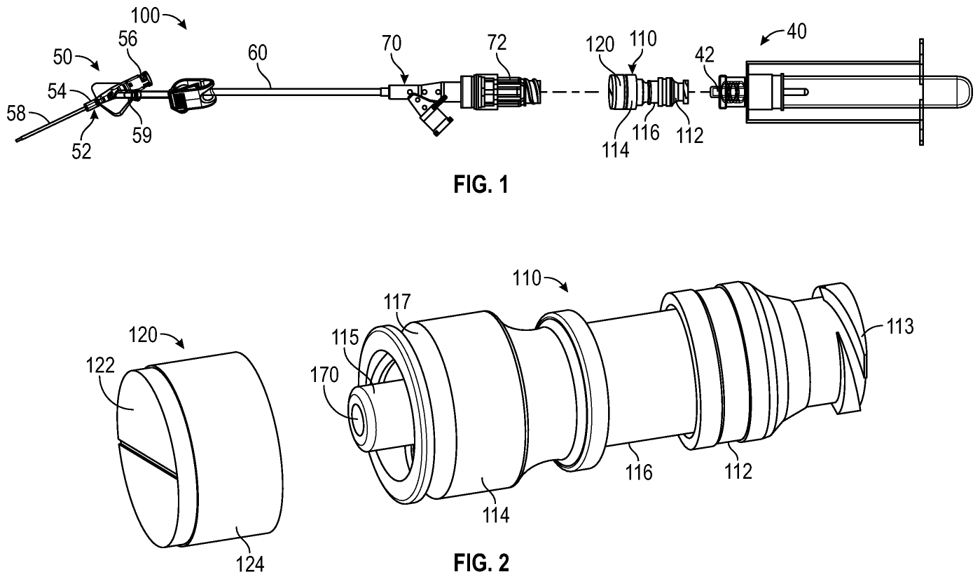

FIG. 2 illustrates an exploded perspective view of the flow restriction device 110, in accordance with some embodiments of the present disclosure. As depicted in FIG. 2, in some embodiments, the flow restriction device 110 may include a proximal housing 112 configured to couple to the blood collection device 40. For example, the proximal housing 112 may be integrated with the blood collection device 40 or monolithically formed with the blood collection device 40 as a single unit. As another example, the proximal housing 112 may include a female luer connector 113, which may be coupled with a male luer connector 42 of the blood collection device 40.

In some embodiments, the flow restrictor device 110 may be in the form of a cylindrical body extending from the proximal housing 112 to the distal housing 114, with the intermediate housing 116 interposed between the proximal and distal housings 112 and 114. An internal flow channel 170 may be disposed through the proximal, intermediate and distal housings 112, 116, 114, providing a fluid flow pathway from end to end of the flow restrictor device 110.

The distal housing 114 may include a male luer connector 115 and a fluid shield assembly 120 coupled to the distal housing 114. The fluid shield assembly 120 may include a split septum 122 coupled to a luer enclosure 124. For example, the split septum 122 may be glued, welded, overmolded or any other suitable coupling to the luer enclosure 124. The luer enclosure 124 is coupled to the distal housing 114. The luer enclosure 124 may be removably coupled to the distal housing 114, such as by a friction fit. As another example, the luer enclosure 124 may be integrally coupled to the distal housing 114 (e.g., glued, welded, overmolded).

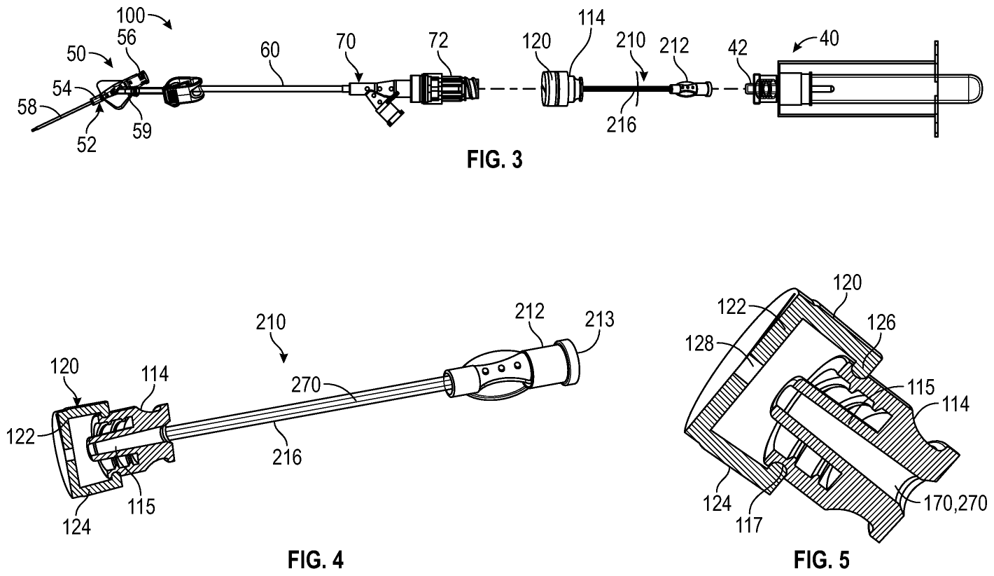

FIG. 3 illustrates the vascular access device 100 including a PIVC assembly 50 that includes a flow restriction device 210 (e.g., an extension set), in accordance with some embodiments of the present disclosure. The flow restriction device 210 may be configured to reduce a likelihood of hemolysis during blood collection using the vascular access device 100. In some embodiments, the flow restriction device 210 may include the distal housing 114, male luer connector 115 and fluid shield assembly 120 described above. In some embodiments, the flow restriction device 210 may be fluidly coupled to the Y-adapter 70. In some embodiments, the flow restriction device 210 may be configured to couple to the proximal end 56 of the catheter hub 52 or another suitable portion of the catheter assembly 50. In some embodiments, the flow restriction device 210 may be coupled directly to the catheter assembly 50, eliminating the extension tube 60 and providing a compact catheter system.

In accordance with some embodiments, the flow restriction device 210 may be configured to couple to a blood collection device 40. For example, the flow restriction device 210 may include a female luer connector 213 at proximal housing 212, which may be coupled with a male luer connector 42 of the blood collection device 40.

FIG. 4 illustrates a partial cross-sectional perspective view of the flow restriction device 210, in accordance with some embodiments of the present disclosure. As depicted in FIG. 4, in some embodiments, the flow restriction device 210 may include a proximal housing 212 configured to couple to the blood collection device 40. For example, the proximal housing 212 may be integrated with the blood collection device 40 or monolithically formed with the blood collection device 40 as a single unit. As another example, the proximal housing 212 may include a female luer connector 213, which may be coupled with a male luer connector 42 of the blood collection device 40.

In some embodiments, the flow restrictor device 210 may be in the form of an IV tube 216 extending from the proximal housing 212 to the distal housing 114, with the IV tube 216 interposed between the proximal and distal housings 212 and 114. An internal flow channel 270 may be disposed through the proximal housing 112, the IV tube 216 and the distal housing 114, providing a fluid flow pathway from end to end of the flow restrictor device 210.

The distal housing 114 may include the male luer connector 115 and the fluid shield assembly 120 may be coupled to the distal housing 114 of flow restrictor device 210. The fluid shield assembly 120 may be removably or integrally coupled to the distal housing 214 in any suitable manner as described above.

FIG. 5 illustrates the fluid shield assembly 120 coupled to the distal housing 114/male luer connector 115 of either flow restrictor device 110 or flow restrictor device 210. The fluid shield assembly 120 may include an engagement rib 126 sized and shaped to be received by a groove 117 of the distal housing 114. The luer enclosure 124 and/or the engagement rib 126 may be formed of a flexible material configured to allow the enclosure to be pushed onto the distal housing 114 until the engagement rib 126 slides into the groove 117. Thus, the fluid shield assembly 120 may be added to or removed from the distal housing 114 as needed.

In some embodiments, the fluid shield assembly 120 may be affixed to the distal housing 114. For example, an adhesive or solvent may be placed in the groove 117 prior to sliding the engagement rib 126 into the groove 117. As another example, the luer enclosure 124 and the engagement rib 126 may be overmolded onto the distal housing 114, where the engagement rib 126 is molded into the groove 117. Here, a separate split septum 122 may be glued, welded, overmolded or any other suitable coupling to the luer enclosure 124. In yet another example, the entire fluid shield assembly 120 may be overmolded onto the distal housing 114. As another example, the engagement rib 126 may be welded (e.g., sonic welding, heat welding) into the groove 117.

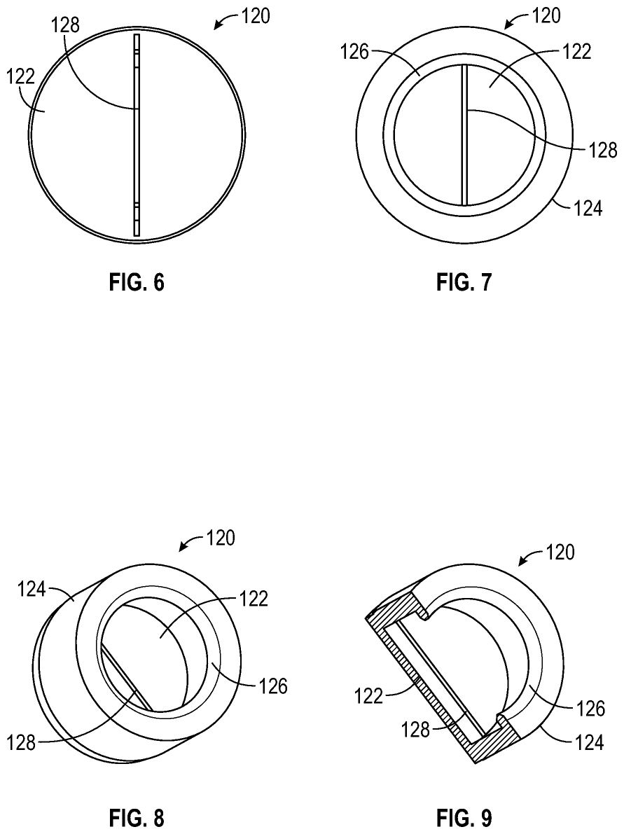

As shown in FIGS. 6-9, the fluid shield assembly 120 may be cylindrical shaped to match the cylindrical shape of a connector (e.g., distal housing 114/male luer connector 115). In aspects of the disclosure, the fluid shield assembly 120 may be sized and shaped to conform with the size/shape of any suitable connector (e.g., oval shaped). The split septum 122 may include a slit 128 and may be formed of any suitable flexible material (e.g., silicone).

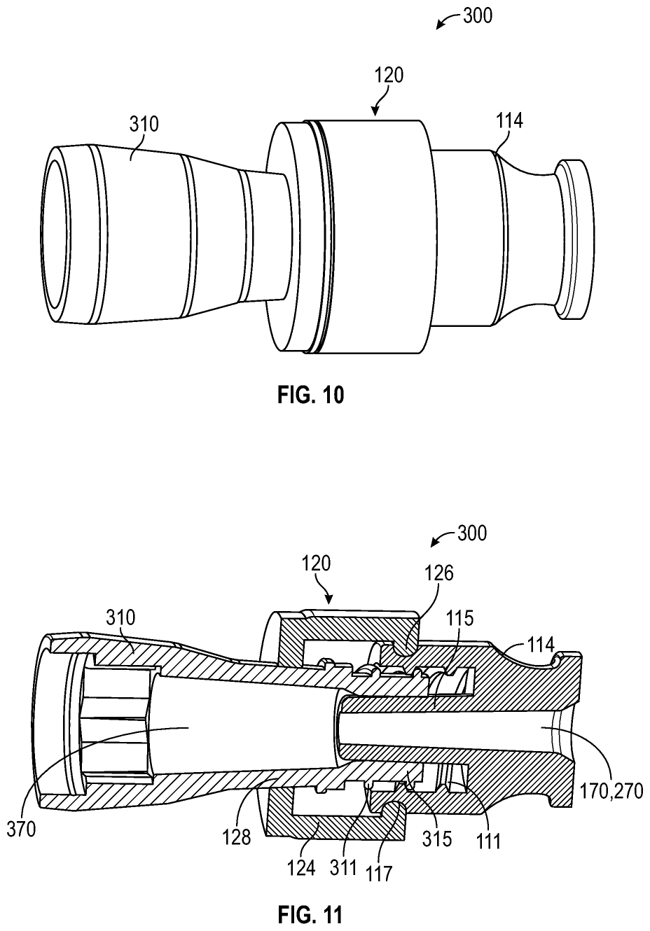

As shown in FIGS. 10 and 11, a connector assembly 300 includes a female connector 310 coupled to the fluid shield assembly 120 and distal housing 114/male luer connector 115 of either flow restrictor device 110 or flow restrictor device 210 shown in FIG. 5. Here, the male luer connector 115 is received within a female luer connector 315 so that the internal flow channel 170, 270 of flow restriction device 110, 210 is fluidly coupled to an internal flow channel 370 of the female connector 310. Also, threads 111 of the distal housing 114/male luer connector 115 are rotatingly engaged with threads 311 of the female connector 310, thus providing a secure connection during use of the connector assembly 300. As can be seen, the slit 128 of the fluid shield assembly 120 has stretched around the outer surface of the female connector 310. In aspects of the disclosure, the female connector 310 may be any suitable female connector configured to mate with the male luer connector 115, such as needle free connector 72 and proximal end 56 of the catheter hub 52, for example.

In use, the fluid shield assembly 120 is coupled to the distal housing 114 such that an end of the male luer connector 115 is enclosed within the luer enclosure 124 and the split septum 122. Any suitable female connector (e.g., proximal end 56, needle free connector 72, female connector 310) may then be coupled to the male luer connector 115. For example, as shown in FIGS. 10 and 11, an end of the female connector 310 is pushed through the slit 128 of the split septum 122, where the flexible material of the split septum 122 provides for the slit 128 to widen so that the flexible split septum 122 stretches and maintains contact with the female connector 310 while the female connector 310 is coupled with the distal housing 114/male luer connector 115. Here, the female connector 310 and the distal housing 114 may each be threaded (e.g., threads 311, 111) and the complete coupling may entail rotating the female connector 310 relative to the distal housing 114 until at least partially or fully seated, thus providing a secure connection.

When disconnection of the flow restriction device 110, 210 is desired, the female connector 310 is uncoupled from the distal housing 114/male luer connector 115 and then the female connector 310 is pulled out through the stretched slit 128 which then contracts back to or close to the size of the slit 128 prior to being connected to the female connector 310. Thus, any fluid (e.g., blood) remaining in the distal housing 114 and/or the lumen of the male luer connector 115 is contained within the luer enclosure 124 of the fluid shield assembly 120. In some aspects of the disclosure, the split septum 122 may function to wipe the end of the female connector 310 as it passes back through the slit 128, thus wiping any fluid from the surfaces of the female connector 310 so that the fluid remains in the fluid shield assembly 120.

In aspects of the disclosure, the fluid shield assembly 120 avoids blood exposure and/or spillage during disconnection of the female connector 310 from the distal housing 114/male luer connector 115. Thus, no secondary action, such as wiping the connectors, needs to be performed by the clinician as any blood drops will be blocked within the luer enclosure 124 and split septum 122 disposed around the distal housing 114/male luer connector 115. In aspects of the disclosure, the split septum 122 self-seals upon removal of the female connector 310.

According to various embodiments of the present disclosure, a method of manufacturing a flow restriction device with a fluid shield assembly may include molding the luer enclosure 124 to the distal housing 114 so that the engagement rib 126 is molded into the groove 117 and/or a portion of the luer enclosure 124 is molded to the outer surface of the distal housing 114. In aspects of the disclosure, the engagement rib 126 may be affixed to the groove 117 via glue or solvent and/or a portion of the luer enclosure 124 may be affixed to the outer surface of the distal housing 114 via glue or solvent. In aspects of the disclosure, the split septum 122 may then be affixed to the exposed end of the luer enclosure 124 by any suitable method, such as gluing, welding or molding. In aspects of the disclosure, the split septum 122 may be affixed to the luer enclosure 124 first to provide an integral fluid shield assembly 120, which may then be affixed to the distal housing 114 as described above. In aspects of the disclosure, the distal housing 114 and luer enclosure 124 may be molded as a single component, and then the fluid shield assembly 120 may be affixed to the luer enclosure 124. In aspects of the disclosure, the distal housing 114, the luer enclosure 124 and the split septum 122 may be molded as a single component in one or more molding operations.

In operation, the flow restriction device 110, 210 with fluid shield assembly 120 may be connected between the catheter assembly 50 and the blood collection device 40 as shown in FIGS. 1 and 3. As such, during blood collection or withdrawal from a patient, blood may flow from the patient's vein into the catheter assembly 50, through the extension tubing 60, enter the internal flow channel 170, 270 of flow restriction device 110, 210 via the distal housing 114, exit the flow restriction device 110, 210 via the proximal housing 112, and enter the blood collection device 40. Accordingly, during blood collection or withdrawal from the patients, the blood may flow into the blood collection device 40 via the internal flow channel 170, 270 having a minimal diameter.

The flow restriction devices 110, 210 of the various embodiments described herein are advantageous over currently existing blood collection systems. For example, during blood draw with currently existing blood draw devices, blood cells may experience shear stress as they flow from a catheter (e.g., catheter system 50) to a blood draw device (e.g., blood collection device 40). The maximum shear stress may be along the wall of the blood cell, often referred to as wall shear stress. Wall shear stress on blood cells is considered a major source of mechanical damage to blood cells causing hemolysis of the blood cells. In some embodiments, the linear internal flow channel 170, 270 having minimal diameter may facilitate increased flow resistance within the vascular access system 100 to distribute the pressure differential and reduce shear stress experienced by the red blood cells of the blood. For example, the minimized diameter of the internal flow channel 170, 270 may provide increased resistance to flow of the blood and thereby decrease blood flow rate within the flow resistance device 110, 210. Since the decreased blood flow rate causes a reduction in shear stress experienced by the red blood cells of the blood, a risk of hemolysis during blood collection may advantageously be reduced.

In addition, the aforementioned configurations of the flow restriction device 110, 210 in which the fluid shield assembly 120 blocks fluid leakage or spillage from the distal housing 114 is advantageous over other blood collection systems in that it is capable of preventing blood spillage from the flow restriction device 110, 210 during disconnection from the blood collection device 40. For example, with other blood collection systems, the recommended blood draw procedures dictate that the blood draw connector should not be disconnected from blood collection device (e.g., luer lock access device (LLAD)) after completion of a blood draw procedure. If the blood draw connector is disconnected from blood collection device, it may cause potential blood spillage from the blood draw connector channel when it is disconnected from blood collection device. In contrast, the fluid shield assembly 120 of the flow restriction devices 110, 210 of the various embodiments described herein in which the split septum 122 blocks fluid from passing from the distal housing 114 provides for the blood to be contained within the fluid shield assembly 120, thereby preventing blood spillage when the flow restriction device 110, 210 is disconnected from the blood collection device 40 for disposal.

The flow restriction devices and associated blood collection systems of the various embodiments described herein additionally provide further advantages over other blood collection systems. For example, add-on flow restriction devices described herein allow for hemolysis-reduction function to be integrated for PIVC blood draw and reduce hemolysis significantly. Further, the flow restriction devices described herein are compatible with PIVC placement and allow for seamless blood draw at insertion. Furthermore, since the flow restriction devices are an add-on which can be easily incorporated without any changes to existing PIVC, there is minimal impact to clinical setting and operations. Additionally, the flow restriction devices described herein minimize or otherwise eliminate the risk of blood spillage during disconnection from the blood draw device. Furthermore, the flow restriction devices described herein have low blood wastage due to a smaller priming volume as compared with other blood collection systems. In addition, the flow restriction devices described herein require less fill time as compared with currently existing blood collection systems.

The subject technology is illustrated, for example, according to various aspects described below. Various examples of aspects of the subject technology are described as examples and do not limit the subject technology. It is noted that an aspect may be combined in any combination with one or more other aspects.

In one or more embodiments, a flow restriction device, comprises: a proximal housing configured to couple to a fluid collection device; a distal housing configured to couple to a catheter assembly; an intermediate housing interposed between the proximal and distal housings; an internal fluid channel extending transversely through the proximal, intermediate and distal housings; and a fluid shield assembly. The fluid shield assembly comprises an enclosure coupled to an end of the distal housing and a split septum coupled to an end of the enclosure, the split septum comprising a flexible material with a centrally disposed slit. The fluid shield assembly is configured to allow connection and disconnection of a female connector to a male connector of the distal housing through the slit of the split septum, and to contain fluid within the enclosure and split septum upon disconnection and withdrawal of the female connector from the male connector.

In aspects of the disclosure, the enclosure of the fluid shield assembly comprises an internal engagement rib configured to couple with a groove on the distal housing. In aspects of the disclosure, the engagement rib is affixed to the groove by one of an adhesive and a solvent. In aspects of the disclosure, the enclosure of the fluid shield assembly is welded to the distal housing. In aspects of the disclosure, the enclosure of the fluid shield assembly is molded to the distal housing. In aspects of the disclosure, the split septum is affixed to the enclosure by one of an adhesive and a solvent. In aspects of the disclosure, the split septum is welded to the enclosure. In aspects of the disclosure, the split septum is molded to the enclosure. In aspects of the disclosure, the flow restriction device is configured to flow blood from the proximal housing to the distal housing and into the fluid collection device, and the fluid collection device comprises a blood collection device. In aspects of the disclosure, the female connector is a needle-free connector of the catheter assembly. In aspects of the disclosure, the proximal housing, the intermediate housing and the distal housing comprise an integral connector assembly having a female connector at the proximal housing and the male connector at the distal housing. In aspects of the disclosure, the intermediate housing comprises an intravenous tube connecting a female connector at the proximal housing and the male connector at the distal housing.

In one or more embodiments, a blood collection system comprises: a blood collection device; a catheter assembly; and a flow restriction device fluidly coupled to the blood collection device. The flow restriction device comprises: a proximal housing coupled to a male connector of the blood collection device; a distal housing comprising a male luer connector configured to couple to a female connector of the catheter assembly; an intermediate housing interposed between the proximal housing and the distal housing; an internal fluid channel extending transversely through the proximal, intermediate and distal housings; and a fluid shield assembly. The fluid shield assembly comprises: a luer enclosure coupled to an end of the distal housing; and a split septum coupled to an end of the enclosure, the split septum comprising a flexible material with a centrally disposed slit, the split septum and the luer enclosure enclosing the male luer connector and an end portion of the distal housing. The fluid shield assembly provides for connection and disconnection of the female connector of the catheter assembly to the male luer connector of the distal housing through the slit of the split septum, and to contain excess blood within the enclosure and split septum upon disconnection and withdrawal of the female connector from the male luer connector.

In aspects of the disclosure, the enclosure of the fluid shield assembly comprises an internal engagement rib configured to couple with a groove on the distal housing. In aspects of the disclosure, the engagement rib is affixed to the groove by one of an adhesive, a solvent, a weld and an over mold. In aspects of the disclosure, the split septum is affixed to the enclosure by one of an adhesive, a solvent, a weld and an over mold. In aspects of the disclosure, the proximal housing, the intermediate housing and the distal housing comprise an integral connector assembly having a female connector at the proximal housing and the male luer connector at the distal housing. In aspects of the disclosure, the intermediate housing comprises an intravenous tube connecting a female connector at the proximal housing and the male luer connector at the distal housing.

In one or more embodiments, a fluid shield assembly for a male luer connector comprises: an enclosure configured to couple to an end of the male luer connector; and a split septum coupled to an end of the enclosure, the split septum comprising a flexible material with a centrally disposed slit, wherein the fluid shield assembly is configured to provide connection and disconnection of a female connector to the male luer connector through the slit of the split septum, and to contain fluid within the enclosure and split septum upon disconnection and withdrawal of the female connector from the male luer connector.

In aspects of the disclosure, the enclosure of the fluid shield assembly comprises an internal engagement rib configured to couple with a groove on an outer surface of the male luer connector.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

A reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

As used herein, the phrase “at least one of” preceding a series of items, with the term “or” to separate any of the items, modifies the list as a whole, rather than each item of the list. The phrase “at least one of” does not require selection of at least one item; rather, the phrase allows a meaning that includes at least one of any one of the items, and/or at least one of any combination of the items, and/or at least one of each of the items. By way of example, the phrase “at least one of A, B, or C” may refer to: only A, only B, or only C; or any combination of A, B, and C.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

It is understood that the specific order or hierarchy of steps, or operations in the processes or methods disclosed are illustrations of exemplary approaches. Based upon implementation preferences or scenarios, it is understood that the specific order or hierarchy of steps, operations or processes may be rearranged. Some of the steps, operations or processes may be performed simultaneously. In some implementation preferences or scenarios, certain operations may or may not be performed. Some or all of the steps, operations, or processes may be performed automatically, without the intervention of a user. The accompanying method claims present elements of the various steps, operations or processes in a sample order, and are not meant to be limited to the specific order or hierarchy presented.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112 (f) unless the element is expressly recited using the phrase “means for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein, but are to be accorded the full scope consistent with the language of the claims and to encompass all legal equivalents. Notwithstanding, none of the claims are intended to embrace subject matter that fails to satisfy the requirement of 35 U.S.C. § 101, 102, or 103, nor should they be interpreted in such a way.

What is claim is:

1. A flow restriction device, comprising:
a proximal housing configured to couple to a fluid collection device;
a distal housing configured to couple to a catheter assembly;
an intermediate housing interposed between the proximal housing and the distal housing;
an internal fluid channel extending transversely through the proximal, intermediate and distal housings; and
a fluid shield assembly comprising:
an enclosure coupled to an end of the distal housing; and
a split septum coupled to an end of the enclosure, the split septum comprising a flexible material with a centrally disposed slit,
wherein the fluid shield assembly is configured to allow connection and disconnection of a female connector to a male connector of the distal housing through the slit of the split septum, and to contain fluid within the enclosure and split septum upon disconnection and withdrawal of the female connector from the male connector.

2. The flow restriction device of claim 1, wherein the enclosure of the fluid shield assembly comprises an internal engagement rib configured to couple with a groove on the distal housing.

3. The flow restriction device of claim 2, wherein the engagement rib is affixed to the groove by one of an adhesive and a solvent.

4. The flow restriction device of claim 1, wherein the enclosure of the fluid shield assembly is welded to the distal housing.

5. The flow restriction device of claim 1, wherein the enclosure of the fluid shield assembly is molded to the distal housing.

6. The flow restriction device of claim 1, wherein the split septum is affixed to the enclosure by one of an adhesive and a solvent.

7. The flow restriction device of claim 1, wherein the split septum is welded to the enclosure.

8. The flow restriction device of claim 1, wherein the split septum is molded to the enclosure.

9. The flow restriction device of claim 1, wherein the flow restriction device is configured to flow blood from the proximal housing to the distal housing and into the fluid collection device, and the fluid collection device comprises a blood collection device.

10. The flow restriction device of claim 1, wherein the female connector is a needle-free connector of the catheter assembly.

11. The flow restriction device of claim 1, wherein the proximal housing, the intermediate housing and the distal housing comprise an integral connector assembly having a female connector at the proximal housing and the male connector at the distal housing.

12. The flow restriction device of claim 1, wherein the intermediate housing comprises an intravenous tube connecting a female connector at the proximal housing and the male connector at the distal housing.

13. A blood collection system, comprising:
a blood collection device;
a catheter assembly; and
a flow restriction device fluidly coupled to the blood collection device, the flow restriction device comprising:
a proximal housing coupled to a male connector of the blood collection device;
a distal housing comprising a male luer connector configured to couple to a female connector of the catheter assembly;
an intermediate housing interposed between the proximal housing and the distal housing;
an internal fluid channel extending transversely through the proximal, intermediate and distal housings; and
a fluid shield assembly comprising:
a luer enclosure coupled to an end of the distal housing; and
a split septum coupled to an end of the enclosure, the split septum comprising a flexible material with a centrally disposed slit, the split septum and the luer enclosure enclosing the male luer connector and an end portion of the distal housing,
wherein the fluid shield assembly provides for connection and disconnection of the female connector of the catheter assembly to the male luer connector of the distal housing through the slit of the split septum, and to contain excess blood within the enclosure and split septum upon disconnection and withdrawal of the female connector from the male luer connector.

14. The blood collection system of claim 13, wherein the enclosure of the fluid shield assembly comprises an internal engagement rib configured to couple with a groove on the distal housing.

15. The blood collection system of claim 14, wherein the engagement rib is affixed to the groove by one of an adhesive, a solvent, a weld and an over mold.

16. The blood collection system of claim 13, wherein the split septum is affixed to the enclosure by one of an adhesive, a solvent, a weld and an over mold.

17. The blood collection system of claim 13, wherein the proximal housing, the intermediate housing and the distal housing comprise an integral connector assembly having a female connector at the proximal housing and the male luer connector at the distal housing.

18. The blood collection system of claim 13, wherein the intermediate housing comprises an intravenous tube connecting a female connector at the proximal housing and the male luer connector at the distal housing.

19. A fluid shield assembly for a male luer connector, the fluid shield assembly comprising:
an enclosure configured to couple to an end of the male luer connector; and
a split septum coupled to an end of the enclosure, the split septum comprising a flexible material with a centrally disposed slit,
wherein the fluid shield assembly is configured to provide connection and disconnection of a female connector to the male luer connector through the slit of the split septum, and to contain fluid within the enclosure and split septum upon disconnection and withdrawal of the female connector from the male luer connector.

20. The fluid shield assembly of claim 19, wherein the enclosure of the fluid shield assembly comprises an internal engagement rib configured to couple with a groove on an outer surface of the male luer connector.
